(19) United States
(12) Patent Application Publication
(10) Pub. No.: US 2008/0109028 A1
(43) Pub. Date: May 8, 2008

(54) Introducer for an Endoluminal Procedure

(76) Inventor: Mikolaj Witold Styrc, Kopstal (LU)

Correspondence Address: WENDEROTH, LIND & PONACK, L.L.P., 2033 K STREET N. W., SUITE 800, WASHINGTON, DC, 20006-1021, US

(21) Appl. No.: 11/631,614

(22) PCT Filed: Jul. 6, 2005
(86) PCT No.: PCT/FR05/01748

§ 371 Date: Oct. 10, 2007

(30) Foreign Application Priority Data
Jul. 7, 2004 (FR) 0407550

(51) Int. Cl. A61M 29/02

(52) U.S. Cl. 606/194

(57) ABSTRACT

The invention relates to an introducer for an endoluminal procedure. The inventive introducer comprises: a guide tube which can be inserted into the human body and which defines an internal guide conduit for an element that is to be introduced, and a hemostatic valve which is formed at one end of the guide tube and which, in the open configuration, defines a passage with a large cross-section in the extension of the guide conduit, and, in the closed configuration, defines a passage with a reduced or zero cross-section. The aforementioned hemostatic valve comprises: a rigid annular support which is connected to the guide tube and which extends axially from same; and an inner annular pouch which is formed axially inside the annular support, which is defined, along the surface thereof facing the axis, by an elastically-deformable flexible membrane, and which is filled with a filling liquid.

The present invention relates to an introducer for an endoluminal procedure, of the type comprising:
a guiding tube which is capable of being introduced in the human body and which delimits internally a guiding channel for an element to be introduced,
a haemostatic valve which is formed at one end of the guiding tube and which has an open state in which it delimits a passage having a large cross-section in the extension of the guiding channel and a closed state in which it delimits a passage having a cross-section which is reduced or zero.

In order to carry out endoluminal procedures, and in particular in order to position endoprostheses which are expandable or self-expandable, or to introduce any other device or instrument, surgeons use a device which is known as an introducer.

The introducer is formed substantially by a guiding tube having a length in the order of one metre or more and an inner diameter of between 1 mm and 8 mm. The guiding tube is intended to be introduced into a vein or an artery of the patient through an incision which is provided at a distance from the region which has to be treated. The guiding tube is introduced until the distal end thereof is in the vicinity of the region to be treated, in particular the region where the stent has to be released. The tube serves to guide the stent and allows it to be released in the body from the distal end of the introducer. A number of filamentary tools are also engaged in the introducer.

After the introducer has been placed in the blood circulation system, the blood under the action of the heart has a tendency to escape through the introducer. In particular, tools which are introduced through the introducer generally have a diameter which is smaller than the inner diameter of the guiding tube so that such blood flow is possible, even in the presence of a tool.

In order to reduce the blood flow, it is known to arrange a haemostatic valve at the proximal end of the guiding tube, that is to say, at the end remaining outside the patient. This valve delimits a hole having a variable cross-section which allows the passage of the tools and which allows the blood flow through the introducer to be reduced and stopped, regardless of whether or not a tool is engaged at that location.

In known introducers, the haemostatic valve is formed by a resiliently deformable flexible sleeve which extends the guiding tube. This sleeve which is produced, for example, from silicone has a length of approximately 5 cm and a diameter corresponding to that of the guiding tube. A clip for clamping the flexible sleeve is provided in order to crush the sleeve in order to reduce the cross-section of the passage delimited by the sleeve by applying the deformed wall of the sleeve against the tool which has been introduced in the tube.

An introducer of this type is not very reliable since the surgeon constantly has to manipulate the clip which clamps the deformable sleeve, each time a tool is engaged in or extracted from the introducer.

The sealing provided by this type of haemostatic valve is thus relatively unsatisfactory.

The object of the invention is to provide an introducer for endoluminal procedures which allows blood flows to be limited in an efficient manner.

To this end the invention relates to an introducer for endoluminal procedures of the above-mentioned type, characterised in that the haemostatic valve comprises:
a rigid annular support which is connected to the guiding tube and which axially extends the tube, and
an inner annular pocket which is formed axially inside the annular support and which is delimited, along the surface thereof directed towards the axis, by a resiliently deformable flexible membrane and is filled with a filling liquid.

According to specific embodiments, the introducer comprises one or more of the following features:
the annular support has an inner diameter of between 1 mm and 10 mm;
the annular support delimits a solid surface and the flexible membrane comprises a flexible tube which is connected, at each end, in a tight manner, to the annular support, the inner pocket being delimited between the annular support and the flexible tube which delimits the membrane;
initially, before being connected to the annular support, the flexible tube which forms the membrane has a nominal inner diameter which is less than 20% of the inner diameter of the annular support and, whilst the flexible tube which forms the membrane is connected to the annular support, the flexible tube which forms the membrane has, in the rest state, at least at the ends thereof, an inner diameter which is substantially greater than the nominal inner diameter;
the nominal inner diameter of the flexible tube is between 0.5 mm and 2 mm;
initially, before being connected to the annular support, the flexible tube which forms the membrane has a nominal inner diameter which is greater than 80% of the inner diameter of the annular support;
the inner diameter of the rigid annular support is between 1.2 and 2 times the diameter of the guiding channel;
the inner pocket has an inflation hole which is capable of being connected to an item of equipment for injecting or removing the inflation fluid in order to vary the volume of the pocket between a reduced volume which corresponds to the open state of the valve and an increased volume which corresponds to the closed state of the valve;
it comprises a syringe which is connected to the inflation hole in order to inject or remove the injection fluid;
it comprises a balloon which is connected to the inflation hole in order to inject or remove the injection fluid;
it comprises a means for isolating the pocket by blocking the inflation hole; and
the pocket delimits a closed space.

The invention will be better understood from a reading of the following description, given purely by way of example, and with reference to the appended drawings, in which:

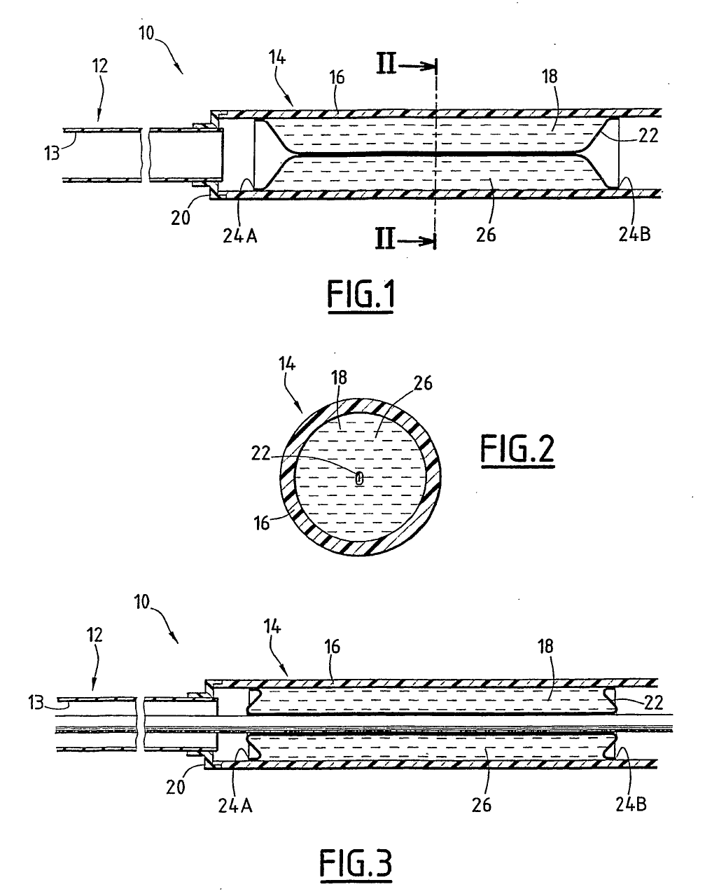

FIG. 1 is a longitudinal section of a first embodiment of an introducer according to the invention, without any tool being engaged through the introducer;

FIG. 2 is a sectional view of the introducer of FIG. 1, taken along the line II-II;

FIG. 3 is an identical view to that of FIG. 1 of the introducer with a tool engaged through the introducer; and

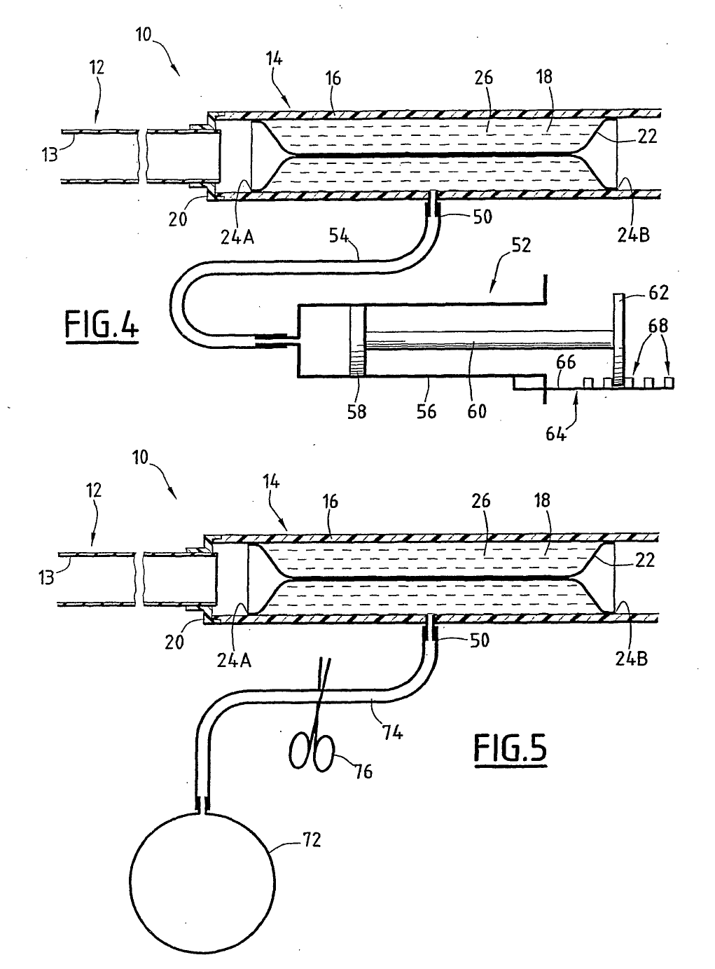

FIGS. 4, 5 and 6 are longitudinal sections of three separate embodiments of the introducer according to the invention, the introducer being illustrated with no tool being engaged.

The introducer 10 illustrated in FIG. 1 substantially comprises a guiding tube 12 and a haemostatic valve 14 which is arranged in the extension of the tube 12 at the proximal end thereof which is intended to be retained outside the body.

The tube 12 has a length of between 20 cm and 150 cm. This length is, for example, in the order of one metre. It delimits a guiding channel 13 which has an inner diameter which is between 1 mm and 8 mm. For reasons of simplification of the drawing, the tube has been truncated in the central portion thereof.

The haemostatic valve 14 comprises a rigid annular support 16 and an inner annular pocket 18 which is formed axially inside the support 16.

The rigid annular support 16 is formed by a non-deformable tube of transparent or metallic polymer material which is arranged coaxially with the tube 12.

The tube 16 has a length of between 2 cm and 15 cm and preferably between 5 cm and 10 cm.

The rigid annular support delimits a solid cylindrical wall. It has an inner diameter which is greater than the inner diameter of the guiding channel 13. This diameter is preferably between 1.2 and 2 times the inner diameter of the guiding channel 13. It is, for example, between 1 mm and 10 mm.

At the connection end thereof, the rigid annular support 16 is connected to the proximal end of the guiding tube 12 by a connection disc 20 which provides sealing between the tubes 12 and 16.

The annular pocket 18 is delimited along the surface thereof directed towards the axis of the support 16 by a resiliently deformable flexible membrane 22.

This membrane is formed, for example, by a flexible tube of latex or silicone. The membrane is connected at each end, by means of adhesive-bonding, to the inner surface of the rigid annular support 16 in order to form peripheral end connections 24A, 24B. These peripheral connections are spaced axially, for example, at a distance of between 3 cm and 7 cm.

A closed space 26 which delimits the pocket 18 is thus formed between the wall of the rigid annular support 16 and the membrane 22. This space 26 is filled with a fluid such as a filling gas or liquid. Preferably, this fluid is a liquid. It is, for example, a physiological serum.

According to a first embodiment, the tube used to form the membrane 22 is constituted, before being connected to the rigid support 16, by a resilient tube having a small diameter, this diameter, in the rest state, preferably being less than 20% of the inner diameter of the annular support 16.

The inner diameter of the tube which forms the membrane 22, before connection, is between 0.5 and 2 mm. In this manner, after connection, the tube is deformed radially by resilient expansion at each of the ends thereof connected to the support 16 in the region of the peripheral connections 24A, 24B.

The quantity of liquid inside the space 26 is selected so that the channel delimited by the pocket 18 is blocked, as illustrated in FIG. 2, the opposite surfaces of the membrane 22 being pressed against each other.

This quantity is selected so that the pressure inside the inner annular pocket 18 is between 1 bar and 5 bar absolute and so that the surfaces of the flexible tube which forms the membrane 22 press against each other over a length of between 50% and 80% of the distance which separates the two peripheral connections 24A, 24B. In this manner, at each end, the membrane 22 forms a conical profile which is convergent from the connections 24A, 24B and which is identical to a funnel.

It will be understood, with an introducer of this type, when no tool is introduced through the haemostatic valve, that the blood flow is stopped by the presence of the inner annular pocket 18 which completely obstructs the rigid annular support 16. In this manner, the blood which can be introduced through the guiding tube 12 is stopped and cannot flow through the completely flattened passage which can be seen in FIG. 2.

When a filamentary tool is introduced, as illustrated in FIG. 3, it displaces the flexible membrane 22, expanding the annular passage which is delimited thereby. This membrane then presses, as illustrated in FIG. 3, precisely on the lateral surface of the tool, conforming exactly to the shape thereof and also preventing in this instance an undesirable flow of blood.

As illustrated in FIG. 3, the deformation of the membrane 22 leads to a reduction in the thickness of the pocket 18. Conversely, the length of the pocket increases so that the pocket is curved at each of the ends thereof.

According to another production variant, the structure of the introducer is as illustrated in FIGS. 1 to 3. However, the flexible tube used to form the membrane 22 is constituted by a tube which, in the rest state, before being assembled, has an inner diameter of between 80% and 100% of the inner diameter of the annular support 16. As above, the ends of the tube are connected to the annular support and the chamber 18 is filled with an inflation fluid. However, in order to allow the tube to deform until its walls are flattened and the cross-section of the channel which it delimits is reduced, the pressure of the fluid in the chamber 26 is relatively high and is, for example, between 10 bar and 20 bar absolute.

In this embodiment, the high pressure provides very satisfactory sealing but requires greater force for introducing the tools through the haemostatic valve.

In the embodiment of FIGS. 4, 5 and 6, elements which are identical or similar to those of the above embodiments have been given the same reference numerals. The haemostatic valve comprises, as in the embodiments illustrated in FIGS. 1 to 3, a rigid annular support 16 and an inner annular pocket 18 delimited by a resiliently deformable flexible membrane 22.

However, the valve further comprises in each case a hole 50 for inflating the pocket 18. This hole is provided through the rigid annular support 16 and allows the pocket 18 to be placed in communication with an item of equipment for injecting or removing the inflation fluid.

In the embodiment of FIG. 4, this equipment is formed by a syringe 52 which is connected to the hole 50 via a catheter 54.

The syringe 52 comprises a body 56 in which a piston 58 equipped with an activation rod 60 is mounted so as to slide in a tight manner.

The rod 60 has, at the end thereof opposite that which is connected to the piston 58, a finger support member 62.

The syringe is provided with a mechanism 64 for immobilising the piston 58. This comprises, for example, a notched rod 66 which is fixedly joined to the body 56 and which extends the body axially. This rod has an assembly of projections 68 which are evenly distributed along the length thereof and which are directed towards the axis of the syringe. These projections are capable of co-operating with the finger support member 62 in order to ensure the axial retention thereof in a plurality of predetermined positions. The rod 66 can be resiliently deformed radially in order to allow the member 62 and the activation piston 58 to be released.

It should be understood that, with a device of this type, the shape of the membrane 22 and the pressure in the chamber 18 can be modified by means of injecting or removing the filling fluid, the fluid being drawn or pushed by the piston 58 of the syringe 52. The immobilisation mechanism of the piston maintains the chamber 18 in a pressurised state.

The action on the syringe 52 is applied by the surgeon during the introduction or removal of a tool in order to ensure satisfactory sealing.

In a variant, the syringe is not provided with the mechanism 64 and the chamber 18 is maintained in a pressurised state by means of clamping the catheter 54 using a clip, or by means of a manually controllable valve which is implanted along this catheter.

In the production variant illustrated in FIG. 5, the syringe 52 is replaced by a balloon 72 which is connected to the hole 50 by means of a catheter 74. The balloon is completely filled with the filling fluid and is normally in communication with the pocket 18. The balloon 72 can be resiliently deformed and is capable of being manually crushed in order to ensure circulation of the filling fluid of the balloon 72 towards the pocket 18. A clamping clip 76 is provided in order to crush the catheter 74 in order to ensure that the chamber 18 is maintained in a pressurised state.

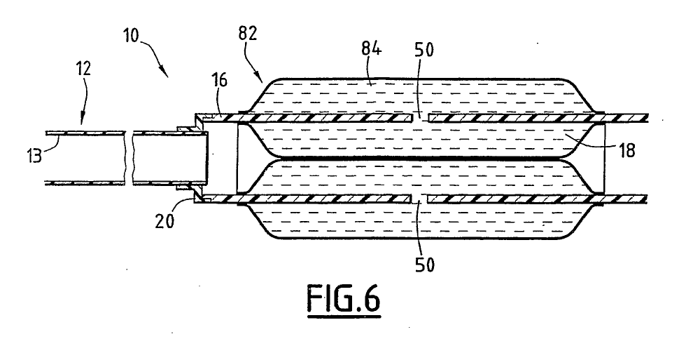

In the embodiment of FIG. 6, the equipment for injecting the inflation fluid is formed by an outer annular pocket 80 which is provided around the rigid annular support 16. This pocket is delimited externally by a deformable membrane 82 which is constituted by a resiliently deformable flexible tube which is arranged around the rigid annular support 16 and which is connected in a tight manner thereto by means of welding or adhesive-bonding at each of the ends thereof.

The inner and outer pockets are in permanent communication via the hole 50. The two pockets 18 and 80 have similar volumes.

In this embodiment, a plurality of holes 50 can be provided along the periphery of the rigid annular support 16.

In order to control the volume of fluid in the pocket and the pressure therein, the surgeon manually applies a pressure around the pocket 80 by placing his hand around it and by pressing the outer pocket radially.

In order to ensure that a pressurised state is maintained in the pocket 18, a toggle (not illustrated) is pressed around the outer pocket 82. This toggle applies a radial pressure to the outer pocket.

1. Introducer for an endoluminal procedure, comprising:
a guiding tube which is capable of being introduced in the human body and which delimits internally a guiding channel for an element to be introduced,
a haemostatic valve which is formed at one end of the guiding tube and which has an open state in which it delimits a passage having a large cross-section in the extension of the guiding channel and a closed state in which it delimits a passage having a cross-section which is reduced or zero,
characterised in that the haemostatic valve Comprises:
a rigid annular support which is connected to the guiding tube and which axially extends the tube, and
an inner annular pocket which is formed axially inside the annular support and which is delimited, along the surface thereof directed towards the axis, by a resiliently deformable flexible membrane and is filled with a filling liquid,
and in that the annular support delimits a solid surface and the flexible membrane comprises a flexible tube which is connected, at each end, in a tight manner, to the annular support, the inner pocket being delimited between the annular support and the flexible tube which delimits the membranes.

2. Introducer according to claim 1, characterised in that the annular support has an inner diameter of between 1 mm and 10 mm.

3. Introducer according to claim 1, characterised in that, initially, before being connected to the annular support, the flexible tube which forms the membrane has, before assembly, in the rest state with no external stresses, an inner diameter which is less than 20% of the inner diameter of the annular support, and in that, whilst the flexible tube which forms the membrane is connected to the annular support, the flexible tube which forms the membrane has, in the rest state, at least at the ends thereof, an inner diameter which is substantially greater than the nominal inner diameter.

4. Introducer according to claim 3, characterised in that the nominal inner diameter of the flexible tube is between 0.5 mm and 2 mm.

5. Introducer according to claim 1, characterised in that, initially, before being connected to the annular support, the flexible tube which forms the membrane has, before assembly, in the rest state with no external stresses, an inner diameter which is greater than 80% of the inner diameter of the annular support.

6. Introducer according to claim 1, characterised in that the inner diameter of the rigid annular support is between 1.2 and 2 times the diameter of the guiding channel.

7. Introducer according to claim 1, characterised in that the inner pocket has an inflation hole which is capable of being connected to an item of equipment for injecting or removing the inflation fluid in order to vary the volume of the pocket between a reduced volume which corresponds to the open state of the valve and an increased volume which corresponds to the closed state of the valve.

8. Introducer according to claim 7, characterised in that it comprises a syringe which is connected to the inflation hole in order to inject or remove the injection fluid.

9. Introducer according to claim 7, characterised in that it comprises a balloon which is connected to the inflation hole in order to inject or remove the injection fluid.

10. Introducer according to claim 7, characterised in that it comprises a means for isolating the pocket by blocking the inflation hole.

11. Introducer according to claim 1, characterised in that the pocket delimits a closed space.
